SEQUENCE LISTING (10 sequences)

SEQ ID NO: 1 - DNA, 18 bp, Artificial Sequence
  primer
ccttgagtcaatacgggt

SEQ ID NO: 2 - DNA, 18 bp, Artificial Sequence
  primer
gctctgacagtaatggca

SEQ ID NO: 3 - DNA, 22 bp, Artificial Sequence
  primer
acccaaagggaagacactacag

SEQ ID NO: 4 - DNA, 22 bp, Artificial Sequence
  primer
tttggctgaggaggtagactac

SEQ ID NO: 5 - DNA, 24 bp, Artificial Sequence
  probe
catgatgatcaaggccaccacagt

SEQ ID NO: 6 - DNA, 753 bp, Homo sapiens
atggccgaaggcggggctagcaaaggtggtggagaagagcccgggaagctgccggagccggcagaggaggaatcccaggttttgcgcggaactggccactgtaagtggttcaatgtgcgcatgggatttggattcatctccatgataaaccgagagggaagccccttggatattccagtcgatgtatttgtacaccaaagcaaactattcatggaaggatttagaagcctaaaagaaggagaaccagtggaattcacatttaaaaaatcttccaaaggccttgagtcaatacgggtaacaggacctggtgggagcccctgtttaggaagtgaaagaagacccaaagggaagacactacagaaaagaaaaccaaagggagatagatgctacaactgtggtggccttgatcatcatgctaaggaatgtagtctacctcctcagccaaagaagtgccattactgtcagagcatcatgcacatggtggcaaactgcccacataaaaatgttgcacagccacccgcgagttctcagggaagacaggaagcagaatcccagccatgcacttcaactctccctcgagaagtgggaggcgggcatggctgtacatcaccaccgtttcctcaggaggctagggcagagatctcagaacggtcaggcaggtcacctcaagaagcttcctccacgaagtcatctatagcaccagaagagcaaagcaaaaaggggccttcagttcaaaaaaggaaaaagacataa

SEQ ID NO: 7 - DNA, 21 bp, Artificial Sequence
  shRNA target sequence
cataacaggtcttcttcatat

SEQ ID NO: 8 - DNA, 19 bp, Artificial Sequence
  primer
gaaggtgaaggtcggagtc

SEQ ID NO: 9 - DNA, 20 bp, Artificial Sequence
  primer
gaagatggtgatgggatttc

SEQ ID NO: 10 - DNA, 21 bp, Artificial Sequence
  probe
caagcttcccgttctcagcct

(19) United States
(12) Patent Application Publication
(10) Pub. No.: US 2015/0065386 A1
(43) Pub. Date: Mar. 5, 2015

(54) METHODS FOR ASSESSING ADJUSTED CANCER STAGE OR PROGNOSIS OF SUBJECT WITH HEPATOCELLULAR CARCINOMA

(71) Applicant: National Cheng Kung University, Tainan City (TW)

(72) Inventor: Chung-Liang Ho, Taipei City (TW)

(21) Appl. No.: 14/533,233

(22) Filed: Nov. 5, 2014

Related U.S. Application Data
(63) Continuation-in-part of application No. 13/672,123, filed on Nov. 8, 2012.

(30) Foreign Application Priority Data
Nov. 8, 2011 (TW) 100140770

(51) Int. Cl. C12Q 1/68; G06F 19/22

(52) U.S. Cl. CPC C12Q 1/6886; G06F 19/22; C12Q 2600/158; C12Q 2600/118; C12Q 2600/112
USPC 506/9; 435/6.12; 435/6.11; 702/19

(57) ABSTRACT

Disclosed herein is a method for assessing an adjusted cancer stage of a subject suffered from hepatocellular carcinoma. The subject has been assigned with a preliminary cancer stage based on the result of a clinical staging and/or pathological staging assessment, and the assessment is made based on both the preliminary cancer stage and the expression level of a cancer stem cell marker gene, Lin-28 homolog B (LIN28B) gene, in the blood of the subject. When the cancer stem cell marker gene is detected in the subject's blood, it is determined that the adjusted cancer stage of the subject is at least one stage advanced than the preliminary cancer stage, whereas when the cancer stem cell marker gene is not detected in the subject's blood, it is determined that the adjusted cancer stage of the subject is the same as the preliminary cancer stage.

### CROSS-REFERENCE TO RELATED APPLICATION

This application is a continuation-in part of co-pending U.S. patent application Ser. No. 13/672,123, filed Nov. 8, 2012, which claims priority to Taiwan patent application No. 100140770, filed Nov. 8, 2011, the entireties of which are incorporated herein by reference.

### SEQUENCE LISTING

This application contains a sequence listing filed in electronic form as an ASCII.txt file entitled SEQUENCE LISTING, created on Oct. 5, 2014 and having a size of 2917 bytes. The content of the sequence listing is incorporated herein in its entirety.

### BACKGROUND OF THE INVENTION

1. Field of the Invention

The present disclosure relates to cancer diagnostics. More particularly, the disclosed invention relates to methods for assessing an adjusted cancer stage and/or prognosis of a subject diagnosed with hepatocellular carcinoma (HCC).

2. Description of Related Art

Hepatocellular carcinoma is the fifth most common solid malignant tumors worldwide and the third leading cause of cancer related death. HCC arises most frequently in patients with inflammatory livers resulting either from hepatitis virus (such as hepatitis B virus or hepatitis C virus) infection or from metabolic disorders or toxic insults (e.g., the ingestion of alcohol or aflatoxin B1). Decisions on the therapeutic modalities are made largely according to the status of tumor growth at the time of diagnosis as well as the expected outcomes of the diseases. Therefore, it is important to identify biomarkers suitable for the diagnosis and/or prognosis of HCC.

The alpha-fetoprotein (AFP) gene and glypican-3 (GPC3) gene are examples of the most commonly used diagnostic markers for HCC. Both the AFP and GPC3 genes are oncofetal genes, and thus are highly expressed in early embryogenesis and then become silent in most adult tissues, but will be reactivated in cancer cells. Therefore, oncofetal genes/proteins tend to be good tumor markers due to low background expression in the adults.

Cancer stem cells (CSCs) or tumor stem cells (TSCs) represent a sub-population of cancer cells that possess self-renewal capacity and ability to generate the heterogeneous lineages of cancer cells through differentiation. Cancer stem cells have been identified in various malignant tumors, including leukemia, brain cancer, breast cancer, colorectal cancer, ovarian cancer, pancreatic cancer, prostate cancer, and hepatocellular carcinoma. These observations have led to the development of the cancer stem cell theory. According to this theory, neoplasms, like body tissues, could be hierarchically organized, and cancer stem cells are at the apex of this cellular hierarchy. Therefore, cancer stem cells are characterized in their ability to recapitulate the generation of a continuously growing tumor. Currently, CSCs in HCC can be identified by several cell surface antigens including c-kit, CD133, CD90, CD44, OV6, and CD326 (EpCAM).

The identification of cancer stem cells is now being pursued actively in many human malignant tumors. However, since cancer stem cells only make up a very small fraction of the total tumor mass, the identification process is often labor-intensive and requires highly skilled artisans for conducting such task. Moreover, to maximize the probability of detecting cancer stem cells, the sampling procedural is best directed to the tumor lesion that involves invasive sampling procedure, which is unfavorable to the patients.

In view of the foregoing, there exists a need in the art of a novel marker and a method for the identification of cancer stem cells. Moreover, the maker is preferably detectable by a minimal-invasive approach. Such marker as well as the detecting method would be an ideal tool for the diagnosis, treatment, and/or prognosis of malignant tumors.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present disclosure is directed to a method for assessing an adjusted cancer stage of a subject suffered from hepatocellular carcinoma based on the presence of circulating cancer stem cells in the subject, as well as a preliminary cancer stage that has been assigned to the subject using a clinical staging and/or pathological staging assessment. For example, the circulating cancer stem cells are of hepatocellular carcinoma origin. According to the principles and spirits of the present disclosure, the method is a non-invasive or minimal-invasive technology that uses samples obtained or derived from body fluid, such as blood, in which the presence of the circulating cancer stem cells is determined based on the detected expression of a novel cancer stem cell marker gene, Lin-28 homolog B gene (LIN28B gene) that has at least 95% nucleic acid sequence identity to the sequence of SEQ ID NO: 6. Further, the present method is capable of qualitatively and quantitatively identifying circulating cancer stem cells, despite the scarcity of circulating cancer stem cells in the body fluid of the subject. The present method is also advantageous in that it is the first to propose that the presence of circulating cancer cells in a subject can be used to assess and adjust the subject's previously assigned cancer stage, and the results confirm that the adjusted cancer stage more accurately reflects the subject's postoperative prognosis.

According to one embodiment of the present disclosure, the method comprises the following steps. First, a blood sample is obtained from the subject. Afterwards, a plurality of mononuclear cells are isolated from the blood sample. Then, a cycle threshold of the LIN28B gene (Ct_{CSC} value) in the plurality of mononuclear cells is determined by a real-time reverse transcription polymerase chain reaction (RQ-PCR)-based process using a first primer pair, comprising a forward primer having the sequence of SEQ ID NO: 3 and a reverse primer having the sequence of SEQ ID NO: 4. Next, the Ct_{CSC} value is compared with a reference value to determine whether the LIN28B gene is expressed in the plurality of mononuclear cells. Specifically, when the Ct_{CSC} value is less than the reference value of 38 and greater than 0, a positive result is concluded, which indicates the expression of the LIN28B gene in the plurality of mononuclear cells; on the other hand, when the Ct_{CSC} value is equal to or greater than the reference value of 38 or the RQ-PCR-based process gives no Ct_{CSC} value, a negative result is concluded, which indicates the lack of expression of the LIN28B gene in the plurality of mononuclear cells. Then, the subject's adjusted cancer stage is determined based on the above-mentioned positive or negative result and the subject's preliminary cancer stage. Specifically, when a positive result is concluded, it is determined that the adjusted cancer stage of the subject is at least one stage advanced than the preliminary cancer stage; whereas when a negative result is concluded, it is determined that the adjusted cancer stage of the subject is the same as the preliminary cancer stage.

According to optional embodiments, the LIN28B gene has at least 98% nucleic acid sequence identity to the sequence of SEQ ID NO: 6. In one example, the LIN28 gene has a sequence identical to the sequence of SEQ ID NO: 6.

In optional embodiment, the method further comprises an evaluating step, which provides a postoperative prognosis of the subject. In this case, a positive result is an indication of an unfavorable postoperative prognosis for the subject, whereas a negative result is an indication of a favorable postoperative prognosis for the subject. In one example, the favorable postoperative prognosis is a recurrence-free survival ≧12 months, and the unfavorable postoperative prognosis is a recurrence-free survival less than 12 months.

Still optionally, the method further comprises an evaluating step in which the postoperative prognosis of the subject is determined based on the adjusted cancer stage determined according to embodiments of the present disclosure.

According to various embodiment of the present disclosure, the blood sample is obtained or derived from the peripheral blood. As could be appreciated, the blood sampling procedure is less invasive as compared to conventional techniques that take tissue samples from the tumor lesion. According to common practice, the blood sample could be used as obtained or processed prior to being sent for the assay.

According to certain embodiment, the plurality of mononuclear cells are isolated by a density gradient technique. In some preferable embodiments, the plurality of mononuclear cells are isolated without using an antibody specific to the epitope of the circulating cancer stem cells.

In some embodiments, the RQ-PCR-based process is a duplex RQ-PCR or a multiplex RQ-PCR.

As could be appreciated, the first primer pair is used for the amplification of the cancer stem cell marker gene, i.e., the LIN28B gene. Optionally, the RQ-PCR process further uses a first fluorescent-labeled probe to detect the presence of the amplified product. For example, the first fluorescent-labeled probe may have a sequence of SEQ ID NO: 5.

To further evaluate the prognosis of the HCC subject, the method further comprises the following steps. A copy number of the cancer stem cell marker gene (Cp_{CSC}) and a copy number of a housekeeping gene (Cp_{HK}) are obtained, respectively. In particular, the Cp_{CSC} value can be determined using the RQ-PCR-based process described above for the determination of the Ct_{CSC} value; whereas the Cp_{HK} value can be determined using an additional RQ-PCR-based process. Then, a relative expression score of the cancer stem cell marker gene to the housekeeping gene is calculated according to equation (1):

Relative Expression Score=log(Cp_{CSC}/Cp_{HK}) equation (1).

The relative expression score is then compared with at least one predetermined cut-off value. For a relative expression score that is equal to or greater than the predetermined cut-off value, it is determined that the subject may have an unfavorable postoperative prognosis. For a relative expression score that is less than the predetermined cut-off value, it is determined that the subject may have a favorable postoperative prognosis.

According to various embodiments of the present disclosure, the housekeeping gene is ACTB gene that encode β-actin or GAPDH gene that encodes glyceraldehyde-3-phosphate dehydrogenase. In the case wherein the housekeeping gene is GAPDH gene, the additional RQ-PCR-based process uses a second primer pair for the amplification of the GAPDH gene. The second primer pair comprises a forward primer having a sequence of SEQ ID NO: 8, and a reverse primer having a sequence of SEQ ID NO: 9. Also, the RQ-PCR-based process may optionally use a second fluorescent-labeled probe that has a sequence of SEQ ID NO: 10 to detect the presence of the amplified product.

In some embodiments, the relative expression score is calculated with respect to GAPDH. For the prognosis of recurrence-free survival, the predetermined cut-off value is −7, and the relative expression score equal to or greater than −7 indicates an unfavorable prognosis of recurrence-free survival, which is less than 12 months; whereas the relative expression score less than −7 indicates a favorable prognosis of recurrence-free survival, which is equal to or greater than 12 months.

In other cases, a predetermined cut-off value of −3 is used for evaluating the prognosis associated with disease-specific survival for the subject. Specifically, the relative expression score equal to or greater than −3 indicates an unfavorable prognosis of disease-specific survival for the subject, whereas the relative expression score less than −3 indicates a favorable prognosis of disease-specific survival for the subject.

According to various embodiments of the present disclosure, the two RQ-PCR-based processes for respectively determining the expression of the cancer stem cell marker (LIN28B) gene and the housekeeping gene can be performed simultaneously. According to certain embodiments, said two RQ-PCR-based processes are performed using a duplex RQ-PCR process or a multiplex RQ-PCR process.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a photograph of a Western blotting results according to one example of the present disclosure;

FIG. 2 is a bar diagram illustrating the side population sizes according to the example of FIG. 1;

FIG. 3 provides a photograph of tumor spheres and a bar diagram illustrating the numbers of tumor sphere according to the example of FIG. 1;

FIG. 4 is a photograph of a Western blotting results according to another example of the present disclosure;

FIG. 5 is a bar diagram illustrating the side population sizes according to the example of FIG. 4;

FIG. 6 provides a photograph of tumor spheres and a bar diagram illustrating the numbers of tumor sphere according to the example of FIG. 4;

FIG. 7 is a bar diagram illustrating the detection limit of the present method according to yet another example of the present disclosure;

FIG. 8 is the standard curve generated from RQ-PCR of LIN28 gene according to one example of the present disclosure;

FIG. 9 is a diagram illustrating the normalized LIN28B expression level of 156 subjects from different experimental group;

FIG. 10 is a diagram illustrating the normalized LIN28B expression level of 96 HCC patients from different experimental group;

FIG. 11 to FIG. 15 are Kaplan-Meier survival plots illustrating the relationship between recurrence-free survival and the presence of circulating LIN28B-expressing cancer cells between various patient subgroups;

FIG. 16 is a Kaplan-Meier survival plot combining the data in FIG. 13 and FIG. 15;

FIG. 17 is a Kaplan-Meier survival plot illustrating the relationship between disease-specific survival and the expression level of LIN28B gene between various patient subgroups.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples and appended claims are collected here. Unless defined otherwise, all technical and scientific terms used herein have the same meaning as commonly understood by one of the ordinary skill in the art to which this invention belongs.

Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The terms “cancer”, “tumor”, and “malignant tumor” are used interchangeably to refer to or describe the physiological condition in mammals that is typically characterized by unregulated cell growth. The term “hepatocellular carcinoma” (HCC) refers to cancer that arises from hepatocytes, as distinct from other types of hepatic cancer that may consist of liver metastases.

Here, the term “subject” or “patient” refers to an animal including the human species that is diagnosed with cancer (e.g., hepatocellular carcinoma) and subject to methods of the present invention. The term “subject” or “patient” intended to refer to both the male and female gender unless one gender is specifically indicated.

As used herein, the term “body fluids sample” refers to any sample isolated or derived from an animal's body fluid. Preferably, the animal may be a human. According to certain embodiments of the present disclosure, body fluid samples include clinical samples derived from subjects in need of medical treatment.

Here, the term “expression level” as applied to a gene refers to the qualitative or quantitative determination of an expression product (or gene product) of said gene. The terms “expression product” and “gene product” are used interchangeably herein to refer to the RNA transcription products (transcripts) of a gene, including mRNA, and the polypeptide translation products of such RNA transcripts. For example, an expression product may be an unspliced RNA, an mRNA, a splice variant mRNA, a microRNA (miRNA), a fragmented RNA, a polypeptide, a post-translationally modified polypeptide, a splice variant polypeptide, etc. Therefore, the expression level may be a determination of the RNA expression level or the polypeptide expression level of the gene. Further, the “expression level” may be an absolute expression level or a relative expression level. According to certain embodiments of the present disclosure, the relative expression level is a “normalized” expression level in which the expression of the marker gene is normalized with respect to the level of an expression product of at least one housekeeping gene (i.e., the reference gene). For example, the expression product of housekeeping gene(s) might be all the measured expression products in the sample, a single reference expression product, or a particular set of expression products.

As used herein, a “sequence” of a nucleic acid refers to the ordering of nucleotides which make up a nucleic acid. Throughout this application, nucleic acids are designated as having a 5′ end and a 3′ end. Unless specified otherwise, the left-hand end of a single-stranded nucleic acid is the 5′ end; and the right-hand end of single-stranded nucleic acid is the 3′ end. The term “downstream” refers to a nucleotide sequence that is located 3′ to a previously mentioned nucleotide sequence. The term “upstream” refers to a nucleotide sequence that is located 5′ to a previously mentioned nucleotide sequence.

“Percentage (%) sequence identity” with respect to any nucleotide sequence identified herein is defined as the percentage of nucleotide residues in a candidate sequence that are identical with the nucleotide residues in the specific nucleotide sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two nucleotide sequences was carried out by computer program Blastn (nucleotide-nucleotide BLAST) provided online by Nation Center for Biotechnology Information (NCBI). The percentage amino acid sequence identity of a given nucleotide sequence A to a given nucleotide sequence B (which can alternatively be phrased as a given nucleotide sequence A that has a certain % nucleotide sequence identity to a given nucleotide sequence B) is calculated by the formula as follows:

$$\frac{X}{Y} \times 100 \%$$

where X is the number of nucleotide residues scored as identical matches by the sequence alignment program BLAST in that program's alignment of A and B, and where Y is the total number of nucleotide residues in A or B, whichever is shorter.

The term “prognosis” is used herein to refer to the prediction of the likelihood that a cancer patient will have a cancer-attributable death or progression, such as recurrence, metastatic spread, and drug resistance, of a neoplastic disease, such as HCC. As could be appreciated, “prognosis” does not refer to the ability to predict the course or outcome of a condition with 100% accuracy. Instead, persons having ordinary skills in the art would understand that the term “prognosis” refers to an increased probability that a certain course or outcome will occur; that is, that a course or outcome is more likely to occur in a subject exhibiting a given condition (e.g., the presence of cancer stem cells), when compared with those subjects not exhibiting the condition. A prognosis is usually made by evaluating factors or symptoms of a disease that are indicative of a favorable or unfavorable course or outcome of the disease. There are many ways that prognosis can be expressed. For example, the prognosis could be expressed in terms of overall survival (OS), recurrence-free survival (RFS), and/or disease-specific survival (DSS). OS is the amount of time from diagnosis or treatment to death. DSS is the amount of time from complete remission to death from HCC, whereas RFS is the amount of time from complete remission to relapse of HCC or death of any cause, whichever comes first.

As used herein, the term “favorable prognosis” refers a prognosis determined for a subject having HCC which is better (i.e., has a more favorable outcome) than the prognosis for a reference subject or group of reference subjects with the same disease. For example, a patient with a favorable prognosis may be expected to exhibit a prolonged OS time or RFS time relative to reference subjects. By contrast, the term “unfavorable prognosis” refers a prognosis determined for a subject having HCC which is worse (i.e., has a less favorable outcome) than the prognosis for a reference subject or group of reference subjects with the same disease. For example, a subject with an unfavorable prognosis may be expected to exhibit a reduced OS time or RFS time relative to reference subjects.

The term “primer” as used herein refers to a single stranded nucleotide sequence which is capable of acting as a point of initiation of synthesis of a primer extension product, when placed under suitable conditions (e.g., buffer, salt, temperature, and pH) in the presence of nucleotides and an agent for nucleic acid polymerization (e.g., a DNA-dependent or RNA-dependent polymerase).

The present disclosure is the first to identify that LIN28B gene is a reliable marker for cancer stem cells, and based (at least in part) on such finding, the present disclosure provides a method for the identification of a rare subpopulation of cancer cells—circulating cancer stem cells.

Lin-28 is an RNA-binding protein, which is first characterized in the nematode, Caenorhabditis elegans, as an important regulator of developmental timing. Mammalian homologs of nematode lin-28 gene (including LIN28 (also known as Lin-28 homolog A, LIN28A) and LIN28B genes) bind to the terminal loop of the precursors of let-7 family miRNAs and block them from being processed into mature microRNAs. Prior studies indicate that LIN28B exhibit oncofetal expression patterns, meaning it is highly expressed in early embryogenic stage and then becomes silent in most adult tissues, but then is reactivated in cancer cells. Also, it is reported that in HCC subjects, LIN28B-expressing tumors are associated with advanced stage(s) and poor clinical outcome (such as a significantly increase in incidence of early recurrence). However, not all oncofetal genes are involved in the maintenance of the stemness of cancer stem cells. Experiments conducted by the present inventor, as described below, reveal that over-expression of LIN28B gene is related to the improved stemness of human HCC cell line. In view of this finding, LIN28B gene may be used as a marker for the identification of cancer stem cells in HCC.

Further, instead of employing conventional methods that detect cancer stem cells in the cancerous tissue, the present disclosure focuses on the identification of cancer stem cells in the circulating body fluid of the subjects. Circulating tumor cells (CTCs) or circulating cancer cells are cells that detached from a primary tumor and circulate into bloodstream. Briefly, during the early stage of tumorigenesis, tumor cells may invade the nearby blood vessels or newly-formed capillaries. These tumor cells often present the cytotoxic CD8 antigen, and are considered foreign by the immune system. Therefore, natural killer (NK) cells would attack them to suppress the accumulation of tumor cells in the circulation. However, some tumor cells (such as aggregates of malignant tumor cells) may escape the immune surveillance, thereby becoming circulating tumor cells. The majority of current technologies for detecting circulating tumor cells involve either use of complex analytic approaches (such as CTC-chip™ or CellSearch™) that detect one or more epithelial cell surface markers (EpCAMs) with specific antibody, or multiplex qPCR approaches for tumor-associated mRNAs. The detection limit of these conventional technologies is about 1 to 10 circulating tumor cells per 1 milliliter of whole human blood. However, the cancer stem cells only account for a minor fraction (about 0.01-2%) of the total tumor mass; and as could be appreciated, in the circulating body fluid, the circulating cancer stem cells are present in an even scarcer amount in relation to large numbers of body fluid cells. Therefore, novel detection strategies are required to detect such extremely low concentrations of circulating cancer stem cells. The present disclosure overcomes this deficiency by the identification of a novel stem cell marker—LIN28B gene. The proposed method aims to provide a reliable approach to identify whether HCC patients have detectable circulating cancer stem cells. Such identification could be used as a diagnostic, treatment, and/or prognostic tool. Validation analysis conducted by the present studies, as discussed below, confirms the efficacy and accuracy of the present method in stratifying HCC patients with respect to the presence or absence of circulating cancer stem cells.

In view of the foregoing, in one aspect, the present disclosure is directed to a method for assessing the adjusted cancer stage of a subject suffered from hepatocellular carcinoma, wherein the subject has been assigned with a preliminary cancer stage based on the result of a clinical staging and/or pathological staging assessment. According to various embodiments of the present disclosure, the method comprises (a) a sampling step, (b) an isolating step, (c) a determining step, (d) a comparing step, and (e) an adjusting step.

In the sampling step (a), a body fluid sample is obtained from the subject. According to various embodiment of the present disclosure, the body fluid sample is obtained or derived from at least one of the following body fluids: peripheral blood, pleural effusion, ascites, cerebrospinal fluid, lymphatic fluid, and bone marrow fluid. In the examples provided below, the body fluid sample is whole blood drawn from HCC patients or other subjects. As could be appreciated, such sampling procedure is less invasive as compared to conventional techniques that involve taking tissue samples from the tumor lesion possibly buried deep inside the body. The conventional blood drawing procedure may be used to collect whole blood from the subject. According to common practice, the body fluid sample could be used as obtained; or processed such as centrifugation, sedimentation, etc., prior to being sent to subsequent assay.

Next, in the isolating step (b), a plurality of mononuclear cells are isolated from the body fluid sample. As could be appreciated, the body fluid sample contains various types of cells, organelles, and subcellular particles. This isolating step aims to enrich the target population for subsequent analysis. In the present instance, the target population is the circulating cancer stem cells which, like lymphocytes, are mononuclear cells. Therefore, any technique capable of separating these mononuclear cells from other components of the body fluid sample is suitable for use in the present method.

According to various embodiments of the present disclosure, the plurality of mononuclear cells are isolated by density gradient separation, which employs a special medium for separating cells, organelles, and other subcellular particles. For example, one commonly used density gradient medium is Ficoll®, a polymer of sucrose with a high synthetic molecular weight. Ficoll® has been traditionally used for separating lymphocytes from other elements in blood.

In conventional methods for detecting circulating cancer stem cells, an antibody against one or more epithelial cell surface markers are often used in conjunction with the density gradient technique, so as to further increase the concentration of the target cells in the sample. Yet, such immunoreaction-based approach is quite labor-intensive and requires highly skilled artisans for conducting such task. The present disclosure, on the other hand, eliminates the need for a marker-specific antibody due to the high detection sensitivity (1 circulating cancer cells per 10⁷ mononuclear cells) of the present method. Therefore, in some embodiments, the plurality of mononuclear cells are isolated without using an antibody specific to the epitope of the circulating cancer stem cells.

After the isolating step (b), the method proceeds to the determining step (c), in which the presence or absence of the expression of the cancer stem cell marker gene in the plurality of mononuclear cells is determined by an RQ-PCR-based process. According to various embodiments of the present disclosure, the expression of the cancer stem cell marker gene is an indication that the body fluid sample contains the circulating cancer stem cells, whereas the lack of expression of the cancer stem cell marker gene is an indication that body fluid sample does not contain the circulating cancer stem cells.

According to various embodiments of the present disclosure, the cancer stem cell marker gene is LIN28B gene or a variant thereof. For example, the LIN28B gene or a variant thereof has at least 95% nucleic acid sequence identity to the sequence of SEQ ID NO: 6. Specifically, the cancer stem cell marker gene may has a nucleic acid sequence identity of 95, 96, 97, 98, 99, or 100% to the sequence of SEQ ID NO: 6.

Specifically, said RQ-PCR process uses a forward primer having a sequence of SEQ ID NO: 3 and a reverse primer having a sequence of SEQ ID NO: 4 for the amplication of the target gene (i.e., LIN28B). Additionally, the RQ-PCR process may optionally use a fluorescent-labeled probe having a sequence of SEQ ID NO: 5 for detecting the amplified target gene. In these cases, the RQ-PCR process comprises assaying the expressed level of the cancer stem cell marker gene in the plurality of mononuclear cells to obtain a cycle threshold of the cancer stem cell marker gene (Ct_{CSC} value).

Next, in the comparing step (d), the Ct_{CSC} value is compared with a predetermined value to determine the presence or absence of the expression of the stem cell marker gene in the plurality of mononuclear cells. According to examples provided below, the predetermined value of 38 has been confirmed to provide conclusive results, while a predetermined value of 36 or 37 fail to render a statistically significant difference between stratified groups.

For a Ct_{CSC} value that is less than 38 and greater than 0, it indicates that the plurality of mononuclear cells (and hence, the body fluid sample obtained from the subject) contain the expression product of the cancer stem cell marker gene (and accordingly, circulating cancer stem cells). For a Ct_{CSC} value that is equal to or greater than 38 or in the case where no Ct_{CSC} value is obtained from the RQ-PCR process, it indicates that the plurality of mononuclear cells (and hence, the body fluid sample obtained from the subject) do contain the cancer stem cell marker gene (and accordingly, circulating cancer stem cells). As apparent from the validation analysis discussed below, this stratification scheme produces subject subgroups that exhibit statistically significant clinical outcomes. Hence, it may serve as a reliable tool for predicting disease outcome, which in term allows treatment and/or medication to be tailored individually; as well as a guide for selecting suitable therapeutic interventions.

Thereafter, in the adjusting step (e), the subject's cancer stage is adjusted based on the results from the comparing step (d) and the preliminary cancer stage that has been assigned to the subject according to the results of conventional clinical staging and/or pathological staging assessment.

Conventional cancer staging can be divided into the clinical staging and the pathological staging. Clinical staging is based on the information obtained from the physical examination, imaging tests (x-rays, CT scans, endoscopy, etc.), tumor biopsies, and blood tests. Clinical staging plays a key role in deciding the best treatment to use for a particular subject. Pathologic staging (also known as surgical staging) is determined further based on information gained by pathological examination of a tumor sample obtained during the surgery to remove the tumor and nearby lymph nodes or for the examination purpose only. Sometimes, a subject's pathologic stage is different from his/her clinical stage (for instance, if the surgery shows the cancer has spread more than it was thought to have spread before surgery), and in this case, the pathological stage is used to predict the prognosis.

The present disclosure is the first to propose that the detected LIN28B gene expression in a subject's blood sample can be used to provide a more precise prognosis, as compared to the conventional clinical staging and/or pathological staging.

For example, for a subject who has been assigned with preliminary cancer stage of stage II according to a clinical staging assessment, a pathological staging assessment, or a combination of both, if it is decided that LIN28B expression is detected in the subject's blood sample according to the present method as described above, in the step (e), the subject's cancer stage is adjusted to stage III.

In another example, if the subject's preliminary cancer stage is stage II, and in step (d) of the present method, it is determined that there is no LIN28B expression in the subject's blood sample, in the step (e), the subject's adjusted cancer stage is still stage II.

In certain optional embodiments, the method further comprises an evaluating step. In particular, these embodiments are suitable for use in the situation where the subject has been diagnosed to have a malignant cancer such as HCC. In this case, the body fluid sample containing the circulating cancer stem cells is an indication of an unfavorable postoperative prognosis for the subject, whereas the body fluid sample not containing the circulating cancer stem cells is an indication of a favorable postoperative prognosis for the subject. For example, the favorable prognosis is a recurrence-free survival equal to or greater than 12 months, and the unfavorable prognosis is a recurrence-free survival less than 12 months.

According to various embodiments of the present disclosure, said evaluating step can be done based on the results of the step (d) or the step (e) described above.

In certain embodiments, the method further comprises a normalizing step in which the expression level of the cancer stem cell marker gene is normalized with respect to the expression level of a housekeeping gene.

For example, the expression levels of the cancer stem cell marker gene and the housekeeping gene are assayed by the RQ-PCR process to obtain a copy number of the cancer stem cell marker gene (Cp_{CSC}) and a copy number of the housekeeping gene (Cp_{HK}). Then, a relative expression score of the cancer stem cell marker gene to the housekeeping gene according to equation (1):

Relative Expression Score=log(Cp_{CSC}/Cp_{HK}) equation (1).

The relative expression score is then compared with at least one predetermined cut-off value. For a relative expression score equal to or greater than the predetermined cut-off value, it indicates an unfavorable postoperative prognosis for the subject. For a relative expression score less than the predetermined cut-off value, it indicates a favorable postoperative prognosis for the subject.

Housekeeping genes are those constantly expressed in certain or all cell types of an organism under normal and diseased physiological conditions. According to various embodiments of the present disclosure, the housekeeping gene may be the ACTB gene encoding β-actin or GAPDH gene encoding glyceraldehyde-3-phosphate dehydrogenase.

In the case where the housekeeping gene is GAPDH gene, the copy number is obtained by performing an RQ-PCR-based process that uses a second primer pair for the amplification of the GAPDH gene. The second primer pair comprises a forward primer having a sequence of SEQ ID NO: 8, and a reverse primer having a sequence of SEQ ID NO: 9. Optionally, a second fluorescent-labeled probe having a sequence of SEQ ID NO: 10 may be used in RQ-PCR-based process to identify the amplified product.

In some embodiments, the relative expression score is calculated with respect to GAPDH. For the prognosis of recurrence-free survival, the predetermined cut-off value is −7, and the relative expression score equal to or greater than −7 indicates an unfavorable prognosis of recurrence-free survival, which is less than 12 months; whereas the relative expression score less than −7 indicates a favorable prognosis of recurrence-free survival, which is equal to or greater than 12 months.

In some other embodiments, the housekeeping is the GAPDH gene, and the relative expression score equal to or greater than −3 indicates an unfavorable prognosis of DSS for the subject, whereas the relative expression score less than −3 indicates a favorable prognosis of DSS for the subject.

According to some embodiments, the present method is capable of detecting the presence of 1 circulating stem cell out of about 10 million (10⁷) mononuclear cells. Such improved detection limit is achieved by the selection of the novel marker for cancer stem cells; that is LIN28B gene.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

### Materials and Methods

Materials

Dulbecco's modified Eagle's medium (DMEM), fetal bovine serum (FBS), penicillin, streptomycin, and TRIzol were obtained from Life technologies (Carlsbad, Calif.). EDTA-K3 was purchased from Becton Dickinson (Franklin Lakes, N.J.). Ficoll-Hypaque density gradient (Histopaque-1077) was commercially obtained from Sigma-Aldrich (Taufkirchen, Germany). Erythrocyte lysis buffer, QIAamp RNA Blood Mini Kit, and RNase-Free DNase I were purchased from QIAGEN (Santa Clarita, Calif.). RNaseOUT RNase inhibitor, SuperScript II Reverse Transcriptase, and Hoechst 33342 dye were commercially obtained from Invitrogen (Carlsbad, Calif.).

Patient Enrollment and Clinical Data

Peripheral blood samples were collected from volunteers recruited at National Cheng-Kung University Hospital (Tainan, Taiwan, R.O.C.). From January 2006 to December 2011, a total of 156 adult patients were enrolled under the approval of the Human Experiment and Ethics Committee of National Cheng-Kung University Hospital with written informed consent of the patients. Among the 156 patients, 96 were diagnosed with primary HCC and underwent hepatectomy at National Cheng Kung University Hospital. Pre-surgery whole blood samples were collected from each participating patients. For comparison, 60 individuals without HCC (non-HCC group) were also included: 31 healthy individuals without any liver disease (healthy group); and 29 patients with viral hepatitis (hepatitis group). In the hepatitis group, 16 patients were diagnosed with HBV, and 13 patients were diagnosed with HCV; among them, 8 patients have cirrhosis.

Recurrence of HCC was documented upon typical findings by computed tomography (CT) or magnetic resonance imaging (MRI) with or without raised serum AFP level or pathological confirmation. Recurrence-free survival (RFS) is defined as time from surgery to the first occurrence of either local or distant recurrence. Disease-specific survival (DSS) is defined as time from surgery to death from HCC.

Peripheral Blood Sample Preparation

Whole blood samples were respectively collected in 10-ml pyrogen-free tubes containing 0.12 ml of 15% K3EDTA and then layered on equal volumes of Ficoll-Hypaque density gradient. After centrifugation at 1600 rpm for 30-40 minutes at 25° C., peripheral blood mononuclear cells (PBMCs) were recovered from the interphase. PBMCs were further washed twice with PBS and centrifuged at 1500 rpm for 5 minutes at 25° C. If the sample volume was less than 3 ml, the whole blood was treated with 5-fold volume of the erythrocyte lysis buffer for 5 minutes at 4° C. The cell pellets were collected and stored at −70° C. for subsequent RNA extraction.

RNA Extraction and PCR-Based Amplification

Total RNA was extracted from cells using the Trizol reagent, and then purified with the QIAamp RNA Blood Mini Kit according to the manufacturer's protocols.

2 μg of total RNA retrieved from the PBCMs was reverse-transcribed into cDNA by 200 units of SuperScript II reverse transcriptase in the reaction buffer containing 500 μg/ml oligo-dT primer, 0.1 M DTT, 40 U/μl RNaseOUT, first-strand buffer, and dNTPs at 42° C. for 50 minutes, and then inactivated by heating at 72° C. for 15 minutes. The cDNA products were stored at −20° C.

For the RT-PCR process, the cDNA was amplified using GeneAmp PCR System 9600 (ABI) with a single 2 minutes initial denaturation at 95° C., followed by touchdown reactions under the following condition: 3 cycles of 94° C. for 30 seconds, 63° C. for 30 seconds, 70° C. for 30 seconds; 3 cycles of 94° C. for 30 seconds, 61° C. for 30 seconds, 70° C. for 30 seconds; 3 cycles of 94° C. for 30 seconds, 59° C. for 30 seconds, 70° C. for 30 seconds; 35 cycles of 94° C. for 30 seconds, 58° C. for 30 seconds, 70° C. for 30 seconds; and final extension at 70° C. for 10 minutes. The PCR products were run on 1.5% agarose gel electrophoresis containing ethidium bromide. RT-PCR primers for GAPDH were purchased from Applied Biosystems. RT-PCR primers for LIN28B were synthesized as designed by LightCycler Probe Design Software 2.0, and the sequences were: the forward primer, CCTTGAGTCAATACGGGT (SEQ ID NO: 1); and the reverse primer, GCTCTGACAGTAATGGCA (SEQ ID NO: 2).

For real-time quantitative RT-PCR(RQ-PCR), 2 μl of cDNA from less than 200 ng of total RNA was then amplified and quantified using a LightCycler system (Roche, Mannheim, Germany). Briefly, the cDNA sample, together with 0.1 μM fluorescence TaqMan probes, 0.5 μM of forward and reverse primers, 2 μl LightCycler TaqMan Master (enzyme:reaction mix=1:3), and PCR-grade water (to a final volume of 10 μl) were used per 20 μl capillary. After centrifugation (LC Carousel Centrifuge 2.0), the capillaries in the sample carousel were amplified with a pre-incubation hold at 95° C. for 10 minutes, followed by 50 cycles of denaturation at 95° C. for 10 seconds, annealing at 55° C. for 30 seconds, and extension at 72° C. for 5 seconds, then cooling at 40° C. for 30 seconds. The expression level of LIN28B was normalized with the expression level of GAPDH.

In the pilot study, Lin28B and GAPDH genes were amplified and detected with commercially available TaqMan® Gene Expression Assays (Applied Biosystems), Catalog numbers: Hs01013729_ml and Hs99999905_ml, respectively. This pilot study was conducted to confirm the efficacy and accuracy of the method and kit provided by the present disclosure.

According to embodiments of the present disclosure, RQ-PCR primers and fluorescence TaqMan probes (the 5′- and 3′-ends were respectively modified with FAM and BHQ) for LIN28B and GAPDH were synthesized as designed by LightCycler Probe Design Software 2.0, and the sequences were:

(SEQ ID NO: 3)
LIN28B, forward primer: ACCCAAAGGGAAGACACTACAG;
(SEQ ID NO: 4)
LIN28B, reverse primer: TTTGGCTGAGGAGGTAGACTAC;
(SEQ ID NO: 5)
LIN28B, probe: CATGATGATCAAGGCCACCACAGT;
(SEQ ID NO: 8)
GAPDH, forward primer: GAAGGTGAAGGTCGGAGTC;
(SEQ ID NO: 9)
GAPDH, reverse primer: GAAGATGGTGATGGGATTTC;
and
(SEQ ID NO: 10)
GAPDH, probe: CAAGCTTCCCGTTCTCAGCCT.

For absolute quantification of the LIN28B gene and the GAPDH gene, the full-length cDNA was cloned and serially diluted over a 7-log range (1 copy to 1,000,000 copies). Then, the diluted samples were subject to RQ-PCR to generate a standard curve. A control sample of 1,000 copies was used in each run, and the amplification result of the target sequence (e.g., LIN28B or GAPDH) in the test sample was compared against the standard curve to provide absolute quantification of the target sequence.

Plasmid Preparation and Retroviral Infection

The PCR product amplified from LIN28B was analyzed by 1.5% agarose gel electrophoresis and extracted by Gel/PCR Fragments Extraction Kit (Geneaid; Sijhih City, Taiwan). The extracted amplicon was ligated to pMSCVpuro vectors (BD Clontech) to obtain the pMSCV-LIN28B vectors which were then transformed into Escherichia coli Top10 competent cells. Plasmids containing the target gene were purified by High-Speed Plasmid Mini Kit (Geneaid; Sijhih City, Taiwan). To further confirm the size of the correct inserts, plasmids digested by specific restriction enzymes were subsequently identified by agarose gel electrophoresis.

Human hepatocellular liver carcinoma cell line (HepG2) was obtained from American Type Culture Collection (ATCC), catalog number: HB-8065 (Manassas, Va.). HepG2 cells were maintained in DMEM supplemented with 10% FBS, 100 U/ml penicillin, and 100 mg/ml streptomycin under 5% CO₂ at 37° C. For the transformed HepG2 cells, 1 mg/ml puromycin (Sigma-Aldrich; St Louis, Mo.) was added to the culture medium to facilitate the expression of LIN28B gene.

Cell transformation was carried out as follows. The pMSCVpuro, and pMSCV-LIN28B vectors were respectively co-transfected into GP2-293T package cells with VSV-G plasmids using calcium phosphate for 48 hours. The HepG2 cell was seeded at a density of 1×10⁶ cells per well in a 6-cm dish and incubated overnight under 5% CO₂ at 37° C. Retroviral supernatant was added with 8 ng/ml of polybrene (Sigma-Aldrich; St Louis, Mo.), and used to infect the HepG2 cell. Pooled HepG2 cell populations expressing either pMSCVpuro or pMSCV-LIN28B were selected with 0.7 μg/mL of puromycin.

shRNA Lentivirus Production

pLKO.1 plasmids expressing small hairpin RNA (shRNA) were purchased from the National RNAi Core Facility Platform (Academia Sinica; Taipei, Taiwan). The lentivirus particles were obtained from the RNAi Core, the Research Center of Clinical Medicine, National Cheng Kung University Hospital. To knock down LIN28B expression, the shRNA of LIN28B (Clone ID: TRCN0000219860, target sequence: 5′-CATAACAGGTCTTCTTCATAT-3′ (SEQ ID NO: 7)) was adopted. A plasmid pLKO_TRC005 was used as the negative control.

Side Population Analysis

1×10⁶ cells challenged with 75 mM verapamil for 30 minutes at 37° C. before the addition of Hoechst 33342 (20 μg/mL, final concentration). Cells were then incubated for 90 minutes in the dark with periodical mixing to dye side population (SP) cells.

Sphere Formation Assay

Cells were seeded on uncoated 6-well culture plates (BD Labware, Bedford, Mass.) in DMEM/F-12 serum-free medium (caisson) containing 1% MEM NEAA, 1× N2, 20 ng/ml EGF, 10 ng/ml bFGF, 100 μg/ml penicillin G, and 100 U/ml streptomycin (Invitrogen, Grand Island, N.Y.). After culturing for 9 days, wells were examined under an inverted microscope at ×20 magnification, and the number of spheres of >50 μm in diameter were counted under a light microscope.

Statistics

For the normalization of LIN28B expression, the LIN28B expression level was divided by the expression level of GAPDH in the same sample. LIN28B mRNA expression in peripheral blood was compared between groups using Wilcoxon rank sum test and was correlated with clinicopathological indicators using chi-square test or Fisher's exact test. RFS and DSS were calculated using the Kaplan-Meier method, and the log-rank test was used to assess the significance of differences between groups. Univariate and multivariate Cox proportional hazards regression model was used to determine the significance of different prognostic factors. Statistical significance was set at P<0.05. The analysis of peripheral blood samples from 96 HCC patients will have 80% power to detect a hazard ratio of 2.2 between LIN28B (+) and LIN28B (−) HCC patients. The proportional hazards assumptions were checked by the martingale and deviance diagnostic plots and no significant deviation from the assumptions of the proportional hazard regression model exists.

### Example I

### LIN28B Relates to Stemness of HCC Cells

Prior researches indicate that LIN28B is an oncofetal gene, and hence, the present invention aims to investigate the relationship between the expression profile of LIN28B and the stemness of cancer cells.

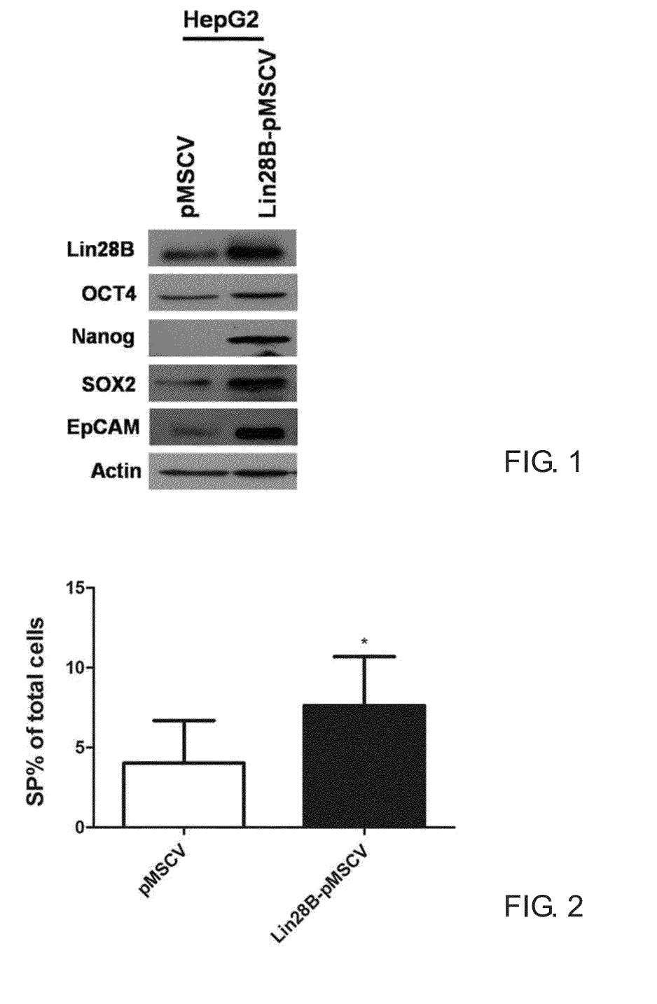

First, HepG2 cell line was transformed by the Lin28B-pMSCV vectors expressing LIN28B gene. The transformed cells were cultured, and then some well-known stem cell markers were analyzed by Western blotting. FIG. 1 is a photograph presenting the results of Western blotting. In comparison to the vector control (pMSCV), it is evident that the stem cell markers OCT4, SOX2, Nanog and EpCAM are respectively upregulated at protein levels by overexpressing LIN28B.

Stem cell-like properties were found in side population (SP) cells in various solid tumors including HCC. The quantification of SP cells revealed that the overexpression of LIN28B gene significantly increased the size of the side population in the HepG2 cell line (FIG. 2).

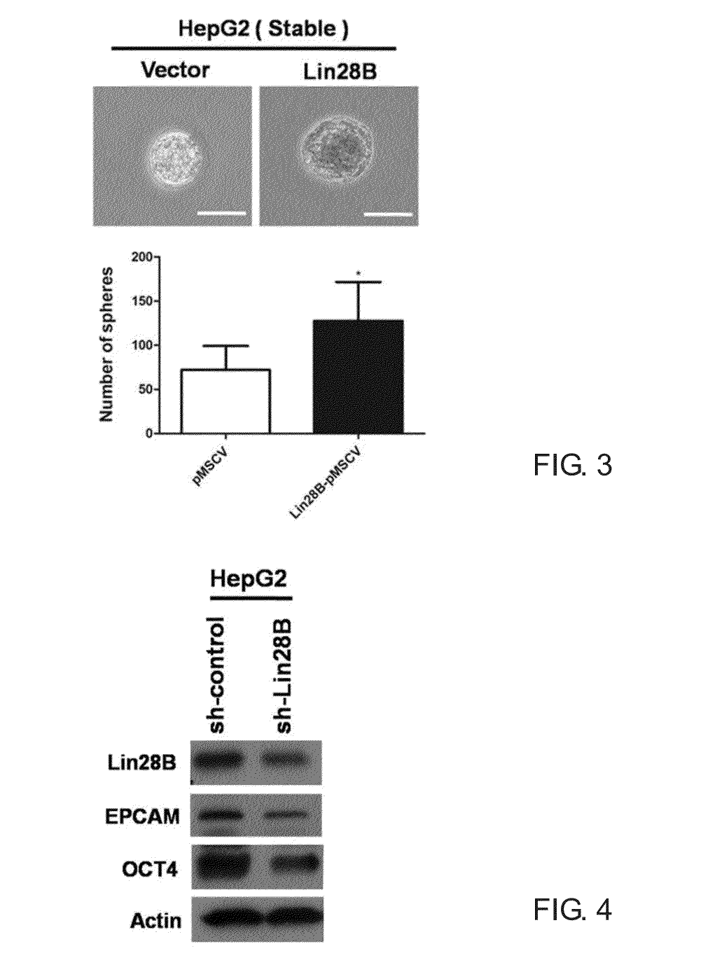

To investigate the effect of LIN28B on tumor sphere-formation, both LIN28B-expressing and control cells were cultured in suspension to generate spheres as an indicator of self-renewal ability in vitro. As depicted in FIG. 3, not only does the number of spheres but also the size increase in LIN28B-expressing HepG2 cells, as compared with that of the control cells.

The foregoing results demonstrate that the enhanced expression of LIN28B gene will indeed promote the self-renewal properties of the HCC cells, and thereby increase the population size or the numbers of these stem cell-like cells.

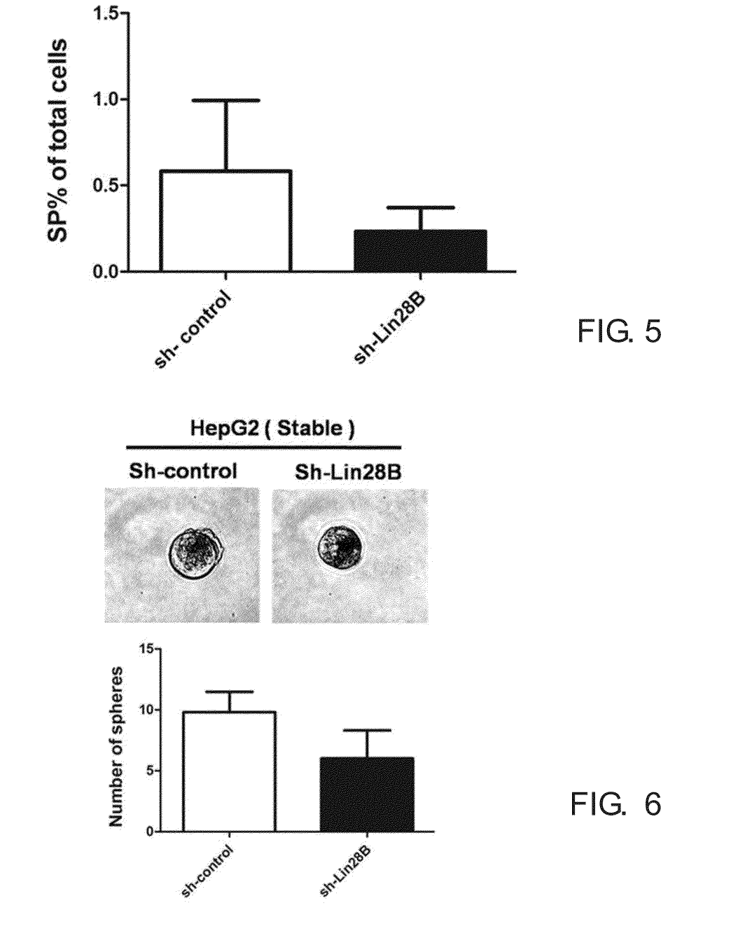

On the other hand, the present disclosure also investigated whether downregulating the LIN28B gene would affect the stemness of HCC cells. Results of Western blotting, as illustrated in FIG. 4, indicate that knocking down LIN28B expression in HepG2 cell line downregulated the stem cell marker expression. However, the different SP size between the sh-control and the sh-LIN28B cells is not statistically significant (P=0.240; FIG. 5). Yet, the reduced expression of LIN28B gene did tend to reduce the formation of tumor spheres (P=0.059; FIG. 6).

Taken together, experiments and analyses in this example indicate that the expression profile of LIN28B gene would affect the stem-like characteristics of HCC cells. Therefore, LIN28B is a potential marker for cancer stem cells.

### Example II

### In Vitro Detection Limit of HCC Cells Pooled in Normal Peripheral Blood

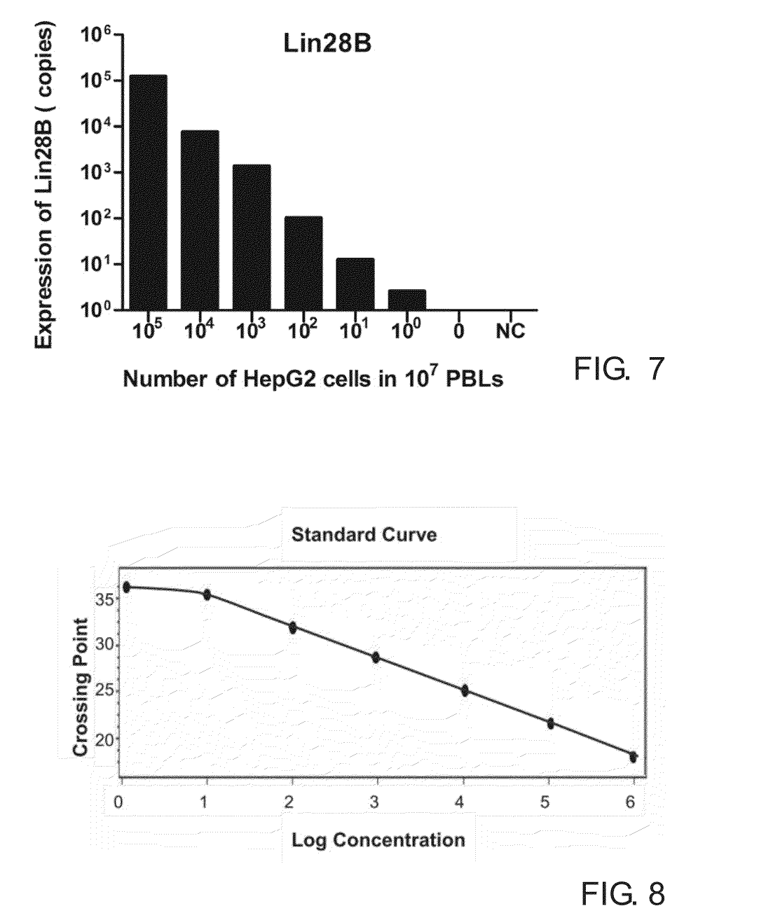

To determine the detection limit of the present method, 1 to 100,000 (10⁵) HepG2 cells were respectively pooled into normal peripheral blood containing 10⁷ peripheral blood leukocytes (PBLs) (about 3 ml whole blood) derived from a healthy donor. RQ-PCR was performed as described above, and the result, as provided in FIG. 7, indicated that LIN28B expression was detected in one HepG2 cell pooled with 10⁷ PBLs with about 5 copies, and was not detected in the normal blood control (NBC) without any HepG2 tumor cell. Further, the mRNA copy numbers of LIN28B gradually decreased upon serial diluting HCC cells. In this example, the detection limit of the present RQ-PCR method is one LIN28B-expressing HepG2 cell per 10⁷ leukocytes in about 3 ml of whole blood. Since the present method is capable of detecting one circulating cancer stem cell in a few milliliters of whole blood, the present method is useful in the clinical setting as a minimal or non-invasive detection approach.

### Example III

### RQ-PCR Standard Curve of LIN28B Gene

RQ-PCR was performed as described above using primers and probes of the present disclosure to obtain the standard curve of LIN28B gene (FIG. 8). In theory, the possible optimum efficiency in PCR is 2, meaning every PCR product is replicated once every cycle. The PCR efficiency calculated from the standard curve of FIG. 8 is 1.958, which is very close to the optimal value. Therefore, the present method may provide reliable and reproducible quantification of LIN28B gene expression.

Further, the reaction was linear from 10 to 10⁶ copies of purified plasmid templates (data not shown), which indicates that for samples containing more than 10 cDNA copies, the present method may reliably detect both LIN28B and GAPDH genes. Also, for the sample containing 10 cDNA copies, the Ct_{CSC} value obtained using the primers and probe of the present disclosure is about 35-36.

### Example IV

### LIN28B is Potential Marker for Detecting Circulating Cancer Stem Cells

RQ-PCR, as described above, was applied to examine the expression of LIN28B in the peripheral blood circulating cells collected from 156 enrolled subject (HCC group, n=96; Healthy group, n=31; and Hepatitis group, n=29). The patient profiles of the 96 HCC patients are summarized in Table 1.

TABLE 1
Variables

| | |
|---|---|
| Mean age, range (years) | 59.13, 27-86 |
| Sex: male/female (cases) | 69/27 |
| Hepatitis virus: B/C/B + C/Non-B Non-C (cases) | 56/24/4/12 |
| Alpha-fetoprotein: median, range (ng/ml) | 14.96, 0.88-68850 |
| Cirrhosis: no/yes (cases) | 50/46 |
| Mean tumor size, range (cm) | 5.35, 1-17 |
| Tumor grade: 1/2/3 (cases) | 14/63/19 |
| Satellite nodule: no/yes (cases) | 76/20 |
| Multifocal tumor: no/yes (cases) | 81/15 |
| Vascular invasion: no/microscopic/major | 49/43/4 |
| branches (cases) | |
| AJCC stage: I/II/IIIA/IIIB/IIIC/IVA (cases) | 37/39/10/3/6/1 |

Tumor differentiation by Edmondson and Steiner grading system.
AJCC, American Joint Committee on Cancer 2010.

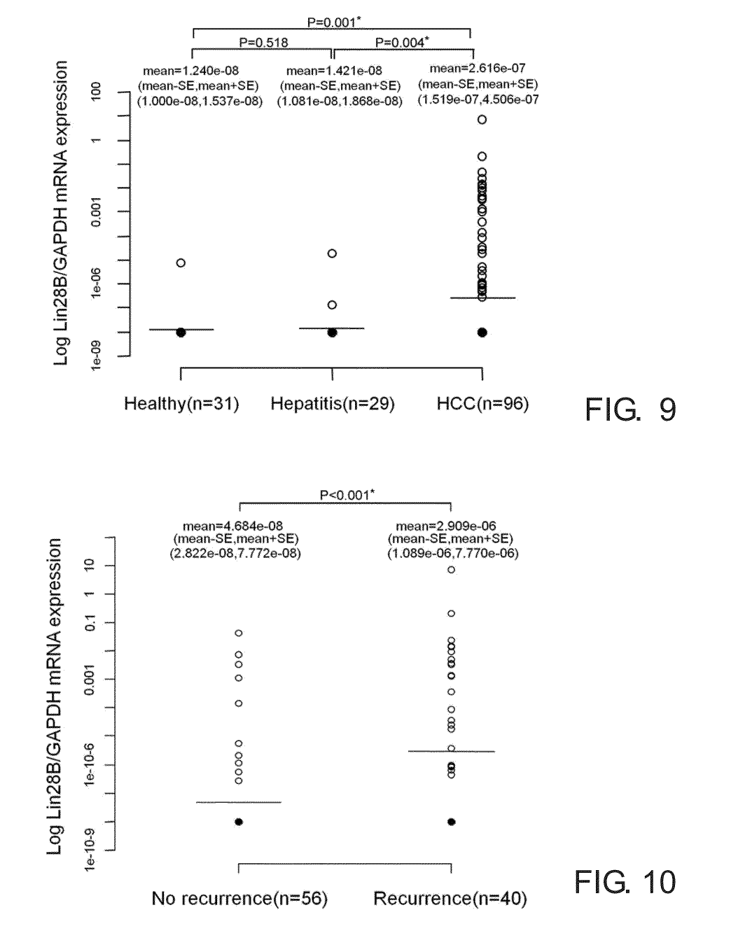

The LIN28B expression profiles among different subject groups are summarized in FIG. 9. In the healthy group, the average normalized LIN28B expression level was 1.240*10⁻⁸±2.685*10⁻⁹, and only one healthy subject had a normalized LIN28B expression significantly higher than the mean value. As to the hepatitis group, the average normalized LIN28B expression level was 1.421*10⁻⁸±3.935*10⁻⁹, and there were 2 hepatitis subjects whose normalized LIN28B expression levels were apparently higher than the mean value. With respect to the HCC group, the average normalized LIN28B expression level was 2.616*10⁻⁷±1.4935*10⁻⁷, and the normalized LIN28B expression levels of 32 HCC subjects were greater than or equal to the average.

Regarding the comparison among different subject groups, there was no statistically significant difference in normalized LIN28B expression levels between the healthy group and hepatitis group (P=0.518), whereas statistically significant differences were observed between the HCC group versus healthy group (P=0.001) and between the HCC group versus hepatitis group (P=0.004).

In sum, on the basis of the detection limit determined in Example II above, LIN28B mRNA was detected in 3 cases (5%) of non-HCC controls (1 in healthy group and 2 in hepatitis group) and in 32 cases (33.3%) of HCC group. As discussed above, LIN28B is an oncofetal gene that is often silenced in differentiated adult cells, except for tumor cells, and hence, we believe most, if not all, of the LIN28B-expressing cells detected by the present method are of cancer cells origin. With respect to the detection of LIN28B expression in the healthy and hepatitis groups, plausible explanation includes the exfoliation of non-malignant regenerate hepatocytes into circulation due to liver inflammation, illegitimate transcription within leukocytes, or actual presence of minimal malignant cells.

The detection results, in combination with the post-operative outcomes of the HCC patients, were further used to verify the prognostic predictive value of LIN28B as a marker for cancer stem cells.

Accordingly, the 96 HCC patients were further divided into two subgroups (the recurrence and non-recurrence groups) based on the recurrence of HCC at the time of the analysis, to investigate whether the LIN28B expression is associated with HCC recurrence. The results, as illustrated in FIG. 10, indicated that in the recurrence group (n=40), the average normalized LIN28B expression level was 2.909*10⁻⁶±3.3405*10⁻⁶, which was almost by 2 orders of magnitude higher as compared to the levels found in the non-recurrence group (4.684*10⁻⁸±2.475*10⁻⁸, n=56). The difference between the two groups is statistically significant (P<0.001). These results indicate that the expression level of LIN28B is positively related to the recurrence of HCC.

Based on results of the above primary analyses, data analysis and computation were subsequently performed to determine a threshold value, which is useful for determining whether the sample contains LIN28B-expressing cells. Two stratification schemes were provided below.

In a first example, results of absolute quantification were used to make such stratification. In this case, the Ct_{CSC} value of each sample is compared with a threshold Ct_{CSC} value, below which the sample is determined to contain LIN28B-expressing cells. After various validation processes with respect to different candidate threshold Ct_{CSC} values, the threshold Ct_{CSC} value is determined to be 38, and a sample is stratified as LIN28B-positive when the Ct_{CSC} value thereof is less than 38 and greater than 0, whereas a sample is stratified as LIN28B-negative when the Ct_{CSC} value is equal to or greater than 38, or when a Ct_{CSC} value could not be determined from the RQ-PCR process (no Ct_{CSC} value).

In a second example, results of relative quantification were used to make such stratification. In this instance, the relative expression score of LIN28B gene to the GAPDH gene was calculated according to equation (1) above. Then, the relative expression scores in each group and subgroup were compared, so as to set up several possible threshold values, and the selected threshold values were than verified by use of the clinical data to evaluate its prediction value. Specifically, the relative expression scores for the healthy group, hepatitis group and HCC groups are −7.9, −7.8, and −6.58, respectively. With respect to the subgroups of HCC subjects, the relative expression scores for the non-recurrence group and recurrence group are −7.32 and −5.5, respectively. After various validation process with respect to different candidate threshold score values, the threshold score value is determined to be −7, and a sample is stratified as LIN28B-positive when the relative expression score thereof is equal to or greater than −7, whereas a sample is stratified as LIN28B-negative when the relative expression score thereof is less than −7.

After stratifying the 96 HCC subjects using the threshold value (e.g., threshold Ct_{CSC} value of 38 or threshold score value of −7) identified above, various clinicopathological indicators of HCC subjects of the LIN28B-positive and LIN28B-negative groups were analyzed. The results summarized in Table 2 are based on the stratification approach using threshold Ct_{CSC} value of 38.

TABLE 2

| | | LIN28B | | |
|---|---|---|---|---|
| Factors | Group | (−) (%) | LIN28B (+) (%) | P-value |
| Age | <60 years old | 35 (70) | 15 (30) | 0.470 |
| | ≧60 years old | 29 (63) | 17 (37) | |
| Sex | Male | 46 (67) | 23 (33) | 1.000 |
| | Female | 18 (67) | 9 (33) | |
| Virus infection | None | 10 (83) | 2 (17) | 0.551 |
| | HBV | 34 (61) | 22 (39) | |
| | HCV | 16 (67) | 8 (33) | |
| | HBV + HCV | 4 (100) | 0 (0) | |
| Cirrhosis | Absent | 28 (56) | 22 (44) | 0.021* |
| | Present | 36 (78) | 10 (22) | |
| Tumor grade | 1-2 | 55 (71) | 22 (29) | 0.046* |
| | 3 | 9 (47) | 10 (53) | |
| Multifocal | Absent | 53 (65) | 28 (35) | 0.551 |
| tumors | Present | 11 (73) | 4 (27) | |
| Satellite nodule | Absent | 51 (67) | 25 (33) | 0.859 |
| | Present | 13 (65) | 7 (35) | |
| Tumor size | <5 cm | 45 (78) | 13 (22) | 0.005* |
| | ≧5 cm | 19 (50) | 19 (50) | |
| Vascular | Absent | 35 (71) | 14 (29) | 0.312 |
| invasion | Present | 29 (62) | 18 (38) | |
| AJCC stage | I, II, IIIA, IIIB | 62 (70) | 27 (30) | 0.044* |
| | IIIC, IVA | 2 (29) | 5 (71) | |
| Serum AFP | <50 ng/ml | 44 (73) | 16 (27) | 0.074 |
| levels | ≧50 ng/ml | 20 (56) | 16 (44) | |

AJCC, American Joint Committee on Cancer 2010.
*P < 0.05.

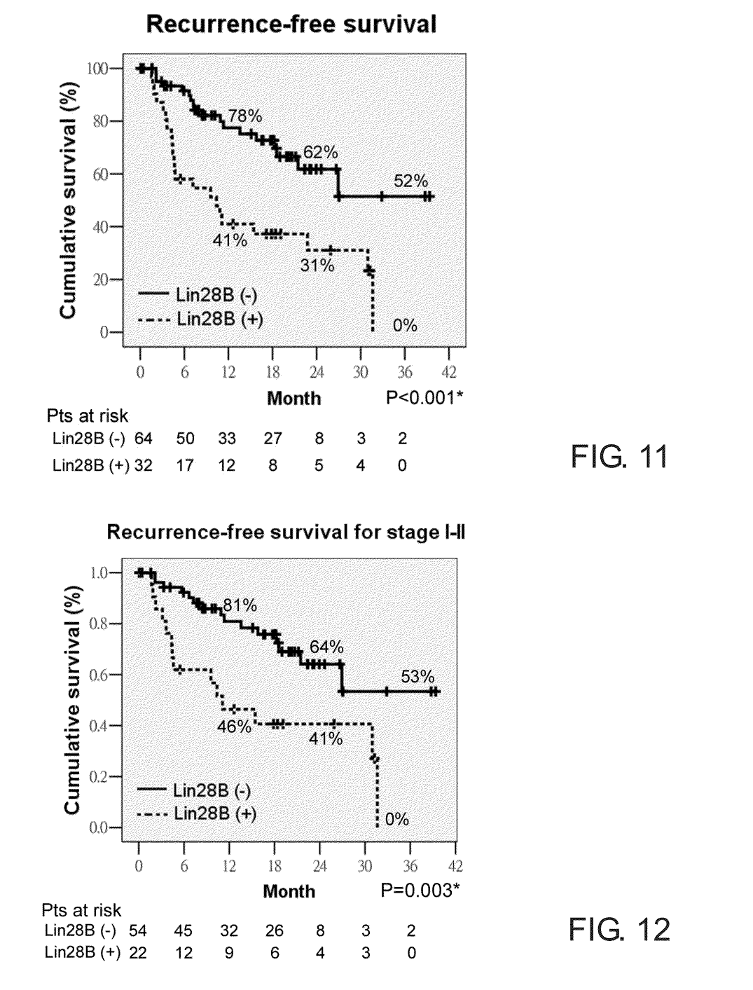

As evident from Table 2, the expression of LIN28B in circulating cells was significantly associated with non-cirrhotic liver (P=0.021), tumor grade (P=0.046), tumor size (P=0.005) and AJCC stage (P=0.044). Results from Kaplan-Meier analysis, as provided in FIG. 11, indicate that the presence of circulating LIN28B-expressing cells is significantly associated with recurrence-free survival (RFS) (P<0.001). Further, for the LIN28B-positive group, the accumulative survival rate of RFS drops to less than 60% within 6 months after the surgery. By contrast, the accumulative survival rate of RFS for in the LIN28B-negative group is greater than 80% at 6 months post-surgery. These findings suggest that the presence of circulating LIN28B-expressing cells is positively related to early HCC recurrence within 6 months.

The HCC subjects were further stratified by AJCC stage, and the results from Kaplan-Meier analysis were provided in FIG. 12 and FIG. 13. For HCC patients with earlier disease stages (stage I to II), the presence of circulating LIN28B-expressing cells significantly correlated with RFS (P=0.003) (FIG. 12). Still referring to FIG. 12, an early recurrence within 6 month is also observed in LIN28B-positive subjects diagnosed with stage I-II HCC for about 40% of subjects in this group, in contrast to about 10% in the LIN28B-negative group. Hence, for subjects diagnosed with stage I-II HCC, the presence of circulating LIN28B-expressing cells discriminates between high and low incidence of early recurrence within 6 months.

To verify the robustness of the threshold Ct_{CSC} value of 38, the same cohort of patients was stratified using the Ct_{CSC} value of 36 or 37 as the threshold Ct_{CSC} value. However, statistical analysis revealed that these two Ct_{CSC} values, when used as the threshold values, failed to produce a statistically significant difference. In contrast, the statistical significance existed only when the Ct_{CSC} value of 38 was used as the threshold value. In view of the foregoing, the threshold Ct_{CSC} value of 38 can be used as a robust threshold for stratifying HCC patients with earlier disease stages (stage Ito II) based on the presence or absence of LIN28B expression in the patient's blood sample.

On the other hand, in more advanced stages (stage IIIA to IVA), the presence of circulating LIN28B-expressing cells did not reach statistical significance in patients (P=0.419) (FIG. 13).

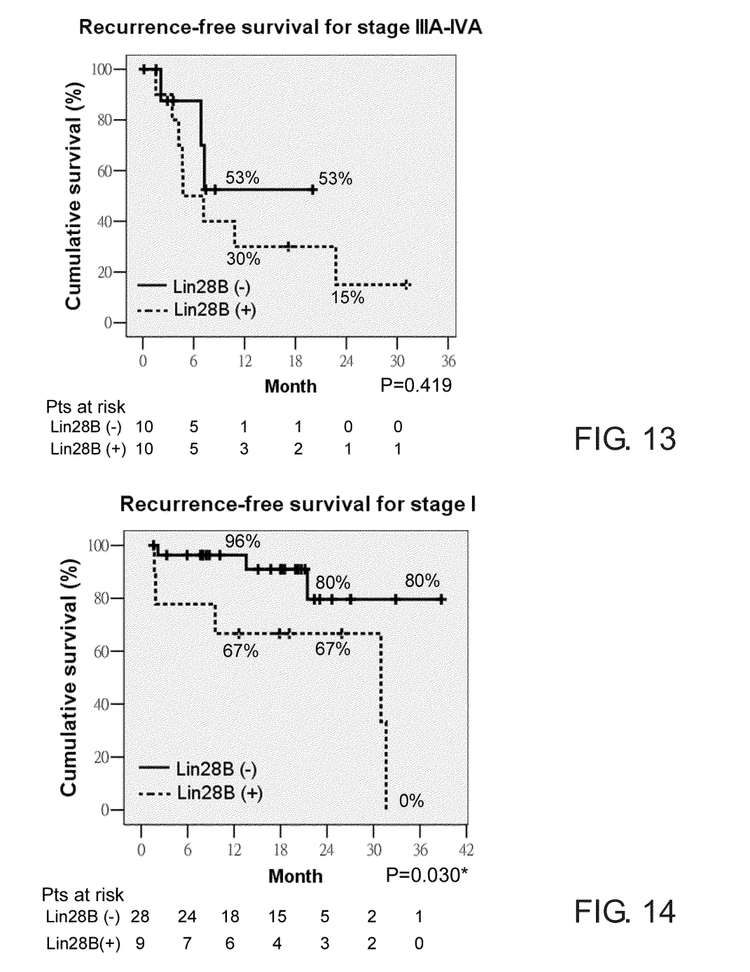

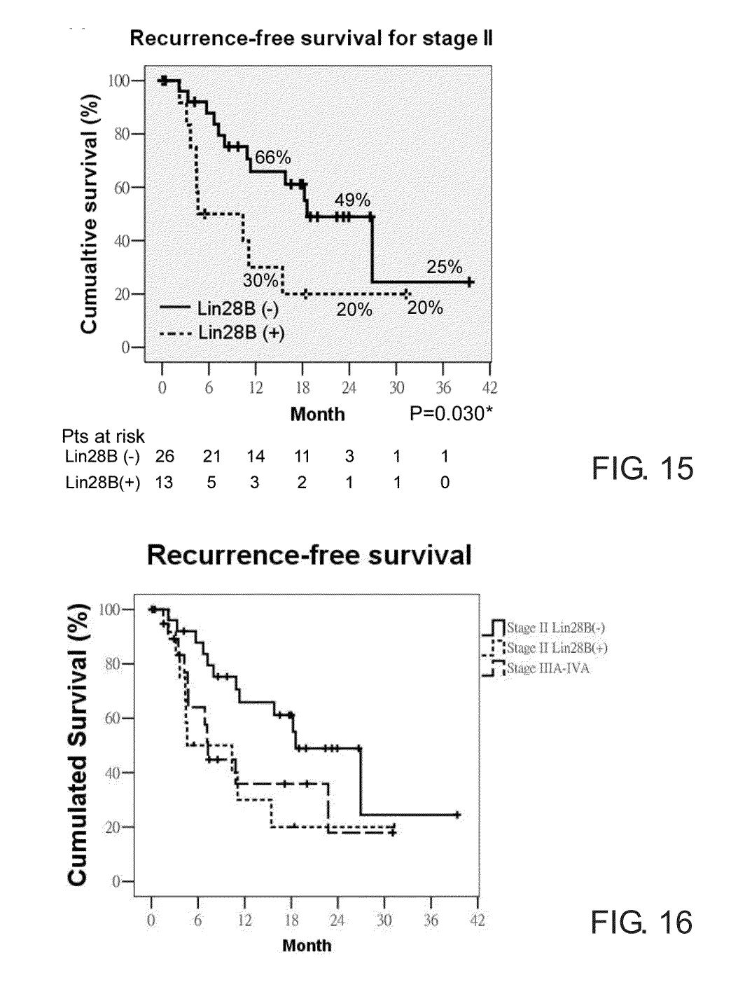

The subjects in stage I-II subgroup were further divided into two subgroups (stage I and stage II), and the results from Kaplan-Meier analysis, as illustrated in FIG. 14 and FIG. 15, indicated that the presence of circulating LIN28B-expressing cells still significantly correlated with RFS in stage I and stage II patients, respectively (P=0.030 and P=0.030, respectively).

By comparing the data in FIG. 14 and FIG. 15, it was found that for stage I HCC patients having LIN28B-expressing cells in the blood, the 12-month RFS rate was 67% (represented by dash line in FIG. 14), which is quite similar to the 12-month RFS rate of stage II HCC patients without LIN28B-expressing cells in the blood (66%; represented by solid line in FIG. 15). Thus, for LIN28B-positive, stage I HCC patients the prognosis may be adjusted to stage II.

Illustrated in FIG. 16 is the recurrence-free survival of all stage IIIA to IVA patients, which is produced by combining the data of two sub-groups provided in FIG. 13. As indicated in FIG. 16, the curve of the recurrence-free survival for stage II HCC patients having LIN28B-expressing cells in the blood overlaps nicely with the RFS curve of stage IIIA to IVA HCC patients. In this case, the prognosis of LIN28B-positivem stage II HCC patients could be adjusted to stage III or IVA.

Accordingly, it is desirable to adjust the HCC patient's cancer stage, which has been determined using conventional clinical staging and/or pathological staging, based on the presence or absence of LIN28B-expressing cells in the patient's blood, so as to more accurately assess the HCC patient's prognosis and propose the suitable treatment.

Univariate analysis was carried out to investigate whether the presence of LIN28B-expressing cells, among other clinicopathological variables, was related to recurrence-free survival (RFS) or recurrence-free survival less than one year (RFS<1 year). The results summarized in Table 3 reveal that circulating LIN28B-expressing cells (P=0.001) and some other variables, such as tumor grade (P=0.047), vascular invasion (P=0.038), and AJCC stage (P<0.001), are significantly associated with decreased RFS.

TABLE 3

| | | RFS | | RFS<1 year | |
|---|---|---|---|---|---|
| Factor | Group | HR | 95% CI | HR | 95% CI |
| Age | <60/≧60 years | 0.647 | (0.341-1.227) | 0.616 | (0.293-1.295) |
| Sex | Male/female | 0.763 | (0.371-1.569) | 0.570 | (0.233-1.396) |
| Viral infection | None/B or C | 2.840 | (0.683-11.809) | 2.080 | (0.494-8.760) |
| | None/Both | 1.313 | (0.118-14.566) | 1.152 | (0.104-12.724) |
| Cirrhosis | −/+ | 0.717 | (0.382-1.344) | 0.576 | (0.104-12.724) |
| Tumor grade | 1-2/3 | 2.081* | (1.009-4.296) | 2.320* | (1.061-5.074) |
| Multifocal tumor | −/+ | 1.644 | (0.722-3.745) | 1.755 | (0.714-4.312) |
| Satellite nodule | −/+ | 1.803 | (0.889-3.654) | 1.454 | (0.647-3.267) |
| Tumor size | <5/≧5 cm | 1.556 | (0.828-2.925) | 1.996 | (0.973-4.095) |
| Vascular invasion | −/+ | 1.961* | (1.039-3.703) | 1.847 | (0.889-3.838) |
| AJCC stage | I/II-IIIB | 3.421* | (1.396-8.385) | 3.782* | (1.521-9.405) |
| | I/IIIC-IVA | 36.3.5* | (3.731-353.236) | 37.631* | (3.860-366.894) |
| Serum AFP | <50/≧50 ng/ml | 1.642 | (0.876-3.076) | 2.457* | (1.198-5.038) |
| Lin28B | −/+ | 2.918* | (1.559-5.463) | 3.637* | (1.749-7.563) |

*P < 0.05.

In the multivariate model, multifocal tumor (P=0.007), AJCC stage (P=0.001) and circulating LIN28B-expressing cells (P=0.043) were independent variables associated with decreased RFS (Table 4). Further, the presence of circulating LIN28B-expressing cells significantly associated with early recurrence less than one year (P=0.035) (Table 4).

TABLE 4

| Factor | Group | HR | 95% CI | HR | 95% CI |
|---|---|---|---|---|---|
| Age | <60/≧60 years | 0.537 | (0.254-1.135) | 0.527 | (0.212-1.307) |
| Sex | Male/female | 0.830 | (0.383-1.798) | 0.655 | (0.250-1.716) |
| Viral infection | None/B or C | 6.089 | (1.094-33.900) | 4.184 | (0.658-26.622) |
| | None/Both | 4.133 | (0.292-58.464) | 3.633 | (0.234-56.348) |
| Cirrhosis | −/+ | 0.546 | (0.250-1.192) | 0.531 | (0.204-1.385) |
| Tumor grade | 1-2/3 | 1.329 | (0.506-3.491) | 1.491 | (0.493-4.512) |
| Multifocal tumor | −/+ | 4.180* | (1.487-11.752) | 3.874* | (1.224-12.249) |
| Satellite nodule | −/+ | 2.166 | (0.900-5.210) | 1.829 | (0.658-5.087) |
| Tumor size | <5/≧5 cm | 0.844 | (0.365-1.951) | 1.064 | (0.420-2.695) |
| Vascular invasion | −/+ | 1.695 | (0.670-4.291) | 1.053 | (0.358-3.101) |
| AJCC stage | I/II-IIIB | 4.987* | (1.261-19.717) | 3.894* | (0.906-16.740) |
| | I/IIIC-IVA | 55.264* | (4.608-662.729) | 49.490* | (3.858-634.778) |
| Serum AFP | <50/≧50 ng/ml | 0.718 | (0.316-1.634) | 1.006 | (0.387-2.614) |
| Lin28B | −/+ | 2.264* | (1.027-4.992) | 2.737* | (1.071-6.993) |

*P < 0.05.

As discussed above, it is inferred that most LIN28B-expressing cells detected by the present method are circulating HCC cells. Theoretically, circulating cancer cells may comprise differentiated tumor cells and/or cancer stem cells. Prior studies indicate that differentiated circulating cancer cells might have limited or negligible proliferation capabilities, often exhibit apoptosis and are unlikely to establish a metastatic lesion at distant sites; whereas circulating cancer stem cells could regenerate the entire population of tumor cells at metastatic sites. In HCCs, it is suggested that circulating cancer stem cells may be homing back to liver and thus contribute to early tumor recurrence. The results in the analyses provided above indicate that presence of LIN28B-expressing cells is associated with poorer postoperative clinical outcomes. For example, on average, LIN28B-positive patients had shorter recurrence-free survival or higher incidences of early recurrence in one year, as compared with that of LIN28B-negative patients. Together with the notion that only circulating cancer stem cells (but not all circulating cancer cells) are responsible for metastasis or HCC recurrence, it is the LIN28B-expressing cells, instead of the differentiated cancer cells, are most likely to be the circulating cancer stem cells. Therefore, according to the principles and spirits of the present disclosure, the detection of the presence of LIN28B-expressing cells in a body fluid (e.g., whole blood) sample of a cancer subject is an indication that the subject has circulating cancer stem cells in his/her circulating system. This information provides valuable insight in the diagnosis, treatment, and/or prognosis of malignant tumor such as HCC.

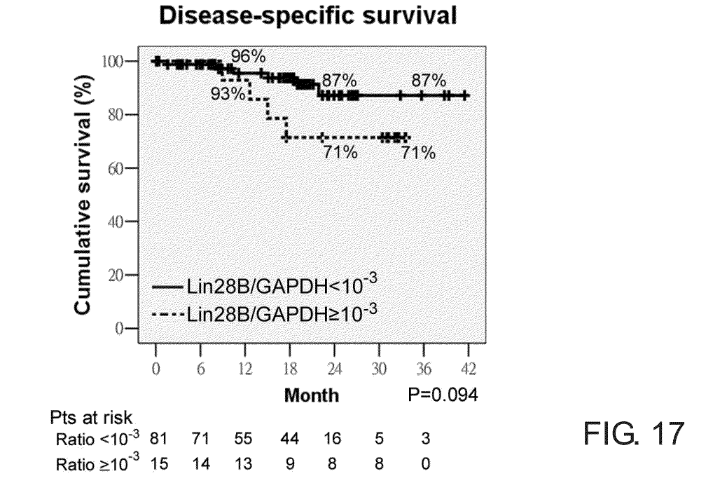

As for disease-specific survival (DSS), multifocal tumor (P=0.046) and AJCC stage (P=0.020) were independent variables in the multivariate analysis (data not shown). However, the presence of circulating LIN28B-expressing cells (identified with a threshold Ct_{CSC} value of 38 or threshold score value of −7) was not relevant with DSS in the multivariate analysis (data not shown). Therefore, an alternative threshold score value for stratification was sought. The result, as illustrated in FIG. 17, indicates that a threshold score value of −3 may achieve a statistically significant stratification. Specifically, for HCC patients having a higher LIN28B expression level (the normalized LIN28B expression level equal to or higher than 10⁻³), the disease-specific survival were shorter than those with a lower LIN28B expression level (P=0.094). This result suggests that the overexpression of LIN28B gene in the circulating cancer stem cells and/or larger amount of circulating cancer stem cells might be associated with a shorter disease-specific survival.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for assessing an adjusted cancer stage of a subject suffered from hepatocellular carcinoma, wherein the subject has been assigned with a preliminary cancer stage based on the result of a clinical staging and/or pathological staging assessment, and the method comprises the steps of,
(a) obtaining a blood sample from the subject;
(b) isolating a plurality of mononuclear cells from the blood sample;
(c) performing a quantitative real-time reverse transcription polymerase chain reaction (RQ-PCR)-based process, using a first primer pair, comprising a forward primer having the sequence of SEQ ID NO: 3 and a reverse primer having the sequence of SEQ ID NO: 4 to determine a cycle threshold of a cancer stem cell marker gene (Ct_{CSC} value) in the plurality of mononuclear cells, wherein the cancer stem cell marker gene is Lin-28 homolog B (LIN28B) gene that has at least 95% nucleic acid sequence identity to the sequence of SEQ ID NO: 6;
(d) determining whether the cancer stem cell marker gene is expressed in the plurality of mononuclear cells based on the Ct_{CSC} value, wherein the Ct_{CSC} value less than 38 and greater than 0 gives a positive result indicating the expression of the cancer stem cell marker gene in the plurality of mononuclear cells, whereas the Ct_{CSC} value equal to or greater than 38 or no Ct_{CSC} value gives a negative result indicating the lack of expression of the cancer stem cell marker gene in the plurality of mononuclear cells;
(e) determining the adjusted cancer stage of the subject based on the result from the step (d) and the preliminary cancer stage of the subject, wherein when the result from the step (d) is positive, it is determined that the adjusted cancer stage of the subject is at least one stage advanced than the preliminary cancer stage, whereas when the result from the step (d) is negative, it is determined that the adjusted cancer stage of the subject is the same as the preliminary cancer stage.

2. The method of claim 1, wherein when the preliminary cancer stage of the subject is stage I and the result from the step (d) is positive, it is determined that the adjusted cancer stage of the subject is stage II.

3. The method of claim 1, wherein when the preliminary cancer stage of the subject is stage II and the result from the step (d) is positive, it is determined that the adjusted cancer stage of the subject is stage III or stage IVA.

4. The method of claim 1, further comprising the step of, evaluating a postoperative prognosis of the subject based on the result from the step (d), wherein a positive result from the step (d) is an indication of an unfavorable postoperative prognosis for the subject, whereas a negative result from the step (d) is an indication of a favorable postoperative prognosis for the subject.

5. The method of claim 4, wherein the favorable postoperative prognosis is a recurrence-free survival equal to or greater than 12 months, and the unfavorable postoperative prognosis is a recurrence-free survival less than 12 months.

6. The method of claim 1, further comprising the step of, evaluating a postoperative prognosis of the subject based on the adjusted cancer stage determined in the step (e).

7. The method of claim 1, wherein the blood sample is obtained or derived from the peripheral blood of the subject.

8. The method of claim 1, wherein the plurality of mononuclear cells are isolated by density gradient separation.

9. The method of claim 1, wherein the RQ-PCR-based process further uses a first fluorescent-labeled probe having a sequence of SEQ ID NO: 5.

10. The method of claim 1, wherein the RQ-PCR-based process is a duplex RQ-PCR or a multiplex RQ-PCR.

11. The method of claim 1, further comprising the steps of,
(f) obtaining a copy number of the cancer stem cell marker gene (Cp_{CSC}) using the RQ-PCR-based process of the step (c);
(g) performing an additional RQ-PCR-based process to assay the expression level of a housekeeping gene in the plurality of mononuclear cells to obtain a copy number of the housekeeping gene (Cp_{HK});
(h) calculating a relative expression score of the cancer stem cell marker gene to the housekeeping gene according to equation (1):
Relative Expression Score=log(Cp_{CSC}/Cp_{HK}) equation (1);
(i) comparing the relative expression score with at least one predetermined cut-off value; and
(j) evaluating a postoperative prognosis of the subject based on the result from the step (i), wherein a relative expression score equal to or greater than the predetermined cut-off value indicates an unfavorable postoperative prognosis for the subject, whereas a relative expression score less than the predetermined cut-off value indicates a favorable postoperative prognosis for the subject.

12. The method of claim 11, wherein the housekeeping gene is a gene encoding β-actin or glyceraldehyde-3-phosphate dehydrogenase (GAPDH).

13. The method of claim 12, wherein the housekeeping gene is the GAPDH gene.

14. The method of claim 13, wherein the predetermined cut-off value is −7, and the favorable postoperative prognosis is a recurrence-free survival equal to or greater than 12 months, whereas the unfavorable postoperative prognosis is a recurrence-free survival less than 12 months.

15. The method of claim 13, wherein the predetermined cut-off value is −3, and the postoperative prognosis is a disease-specific survival for the subject.

16. The method of claim 13, wherein the additional RQ-PCR-based process uses a second primer pair, comprising a forward primer having the sequence of SEQ ID NO: 8 and a reverse primer having the sequence of SEQ ID NO: 9, to assay the expression level of the GAPDH gene in the plurality of mononuclear cells.

17. The method of claim 16, wherein the additional RQ-PCR-based process further uses a second fluorescent-labeled probe having a sequence of SEQ ID NO: 10.

18. The method of claim 11, wherein the RQ-PCR-based process of the step (c) and the additional RQ-PCR-based process of the step (g) are performed concurrently.

19. The method of claim 11, wherein the RQ-PCR-based process of the step (c) and the additional RQ-PCR-based process of the step (g) are performed using a duplex RQ-PCR process or a multiplex RQ-PCR process.
